(19) United States
(12) Patent Application Publication
(10) Pub. No.: US 2025/0054598 A1
(43) Pub. Date: Feb. 13, 2025

(54) USE OF FIELDS BOUNDING BOX TO REDUCE DOSE CALCULATION COMPUTATIONAL EFFORT

(71) Applicant: Siemens Healthineers International AG, Steinhausen (CH)

(72) Inventor: Pierre LANSONNEUR, Lyon (FR)

(21) Appl. No.: 18/233,039

(22) Filed: Aug. 11, 2023

(51) Int. Cl. G16H 20/40; A61N 5/10

(52) U.S. Cl. CPC G16H 20/40; A61N 5/1031

(57) ABSTRACT

Systems and methods for using a field bounding box to reduce the dose calculation volume in a treatment plan optimization process and thereby reduce the computation effort at each iteration of the optimization process.

### FIELD

The present disclosure relates generally to optimizing radiation therapy treatment plans, and more particularly, to systems, methods, and devices for reducing the computational effort in a treatment plan optimization process by reducing the volume for which dose distribution is calculated at each iteration of the optimization process.

### BACKGROUND

Radiation therapy involves medical procedures that use external radiation beams to treat pathological anatomies (tumors, lesions, vascular malformations, nerve disorders, etc.) by delivering prescribed doses of radiation (X-rays, gamma rays, electrons, protons, and/or ions) to the pathological anatomy, while minimizing radiation exposure to the surrounding tissue and critical anatomical structures.

In general, a full radiotherapy planning and treatment workflow includes several phases: a treatment planning phase, a treatment delivery phase, and a monitoring and evaluating phase in which the progress of the treatment, e.g., the dose accumulation is monitored.

In the treatment planning phase, first a precise three-dimensional (3D) map of the anatomical structures in the area of interest (head, body, etc.) is constructed using any one of (or combinations thereof) a computed tomography (CT), cone-beam computed tomography (CBCT), magnetic resonance imaging (MRI), positron emission tomography (PET), 3D rotational angiography (3DRA), or ultrasound techniques. This determines the exact coordinates of the target within the anatomical structure, namely, locates the tumor or abnormality within the body and defines its exact shape and size. This is followed by a prescription step, where a motion path for the radiation beam is computed to deliver a dose distribution to the target within a treatment volume that the radiation oncologist finds acceptable, considering a variety of medical constraints. Treatment volume as used herein, refers to the entire volume that will be subjected to radiation. Then, a team of specialists develop a treatment plan using special computer software to optimally irradiate the tumor and minimize dose to the surrounding normal tissue by designing beams of radiation to converge on the target area from different angles and planes.

In the treatment delivery phase, the radiation treatment plan is executed. During this phase, the radiation dose is delivered to the patient according to the prescribed treatment plan. Generally, a treatment plan is delivered to the patient over a series of radiation treatments referred to as fractions. There are many factors, however, such as, differences in a patient's setup position, changes that might occur if a patient's tumor regresses or if the patient loses weight during therapy, and uncertainties introduced by motion, for example, that can contribute to differences between the prescribed radiation dose distribution and the actual dose delivered (i.e., the actual dose delivered to the target during the radiation treatment). These anatomical and physiological changes can cause the target volumes and surrounding anatomical structures and organs to move and change in size and shape during the therapy. As such, executing or continuing to execute the initial treatment plan may result in an actual received dose distribution that differs from the planned distribution, and thus reduced doses to target volumes and/or increased doses to organs at risk (OARs). During the treatment delivery phase, therefore, the treatment plan may be adapted to the image of the day to better reflect the current situation. This involves making modifications to the initial treatment plan to match the new location and shape of the target volume and surrounding anatomical structures based on subsequently acquired image data.

Generating an optimal treatment plan, whether it is the initial treatment plan generated during the treatment planning phase, or the adapted plan generated during the treatment delivery phase of an adaptive treatment workflow, can be time consuming and tedious. Although some of the steps involved in the plan generation and plan adaptation process have been automated to assist and reduce the workload on the clinical user, it remains a difficult, complex, and time consuming process, especially in the field of intensity modulated radiation therapy (IMRT) and volumetric-modulated arc therapy (VMAT) where complex matrix manipulations are required for calculating and optimizing treatment plans.

Optimization is an iterative process where the user attempts to specify planning goals in the form of dose or biological objectives to create an ideal dose to target structures/target volumes and minimize the dose to critical structures (i.e., organs at risk OARs). During optimization, the dose distribution within the patient is evaluated at each iteration until a satisfactory dose distribution is obtained. Computing the dose distribution at each iteration is, however, a very slow process mainly due to the extent of the volume in which the dose distribution is being evaluated.

Although the volume in which dose distribution is being evaluated has been reduced from the full patient volume to the treatment volume, the computation time is still very slow since the treatment volume can be much larger than the extent of the plan dose distribution volume.

There is thus a need for a plan optimization system and method where the size of the volume for which dose calculation/evaluation is done (i.e., dose calculation volume) is reduced to its minimum.

### SUMMARY

Systems and methods are disclosed to reduce the computation time for dose calculation at each iteration of the treatment plan optimization process by reducing the volume for which dose calculation/evaluation is performed.

Systems and methods are also disclosed to reduce the size of the dose calculation volume to its minimum at each radiation field of the treatment plan.

Systems and methods are also disclosed to calculate dose distribution individually for each radiation field, and to sum the individual dose fields to obtain the dose distribution for the full treatment plan.

In embodiments, a method for optimizing a treatment plan for delivering a radiation dose to a treatment volume within a patient from one or more radiation fields is disclosed, comprising: obtaining, for each radiation field, a reduced volume for which dose distribution is calculated; calculating a dose distribution for each reduced dose calculation volume; and calculating a dose distribution for the treatment plan by summing the calculated dose distributions for each individual radiation field.

In embodiments, the obtaining of a reduced volume for a radiation field includes applying a corresponding field bounding box to crop the treatment volume at each field.

In embodiments, the treatment volume is a body structure containing at least a target volume and an organ at risk, and the cropping includes defining the corresponding field bounding box to enclose the target volume and cropping the treatment volume at the intersection of the field bounding box with the body structure.

In embodiments, the calculating of the dose distribution for a cropped treatment volume includes calculating absorbed dose of radiation at each voxel included in the cropped treatment volume.

Systems including a computer processing device configured to execute a sequence of programmed instructions embodied on a computer-readable storage medium, the execution thereof causing the system to execute the method steps disclosed herein, are also disclosed.

A non-transitory computer-readable storage medium upon which is embodied a sequence of programmed instructions for optimizing a radiation treatment plan by reducing the size of the volume for which dose distribution is calculated at each dose field, including cropping of the treatment volume at each radiation field using a corresponding field bounding box, calculating the dose distribution for each cropped treatment volume, and adding the individual dose distributions to obtain the dose distribution for the treatment plan, and a computer processing system that executes the sequence of programmed instructions embodied on the computer-readable storage medium are also disclosed. Execution of the sequence of programmed instructions can cause the computer processing system to execute the treatment planning and optimization processes described herein.

Objects and advantages of embodiments of the disclosed subject matter will become apparent from the following description when considered in conjunction with the accompanying drawings.

### BRIEF DESCRIPTION OF DRAWINGS

FIG. 1 is a simplified schematic diagram of a radiation therapy system, according to various embodiments of the disclosed subject matter.

FIG. 2 is a schematic illustration of a treatment volume on a planning image, according to various embodiments of the disclosed subject matter.

FIG. 3 is a schematic illustration of a three-beam treatment planning scheme, according to various embodiments of the disclosed subject matter.

FIG. 4A is a schematic illustration of dose distribution grid superimposed on top of a treatment volume, according to various embodiments of the disclosed subject matter.

FIG. 4B is a schematic illustration of a dose calculation volume, according to various embodiments of the disclosed subject matter.

FIGS. 5A and 5B are illustrations of cropping processes using field bounding boxes corresponding to single radiation field treatment plans, corresponding to different radiation fields, according to various embodiments of the disclosed subject matter.

FIG. 5C is an illustration of a cropping process using field bounding boxes in a radiation plan using two different radiation fields, according to various embodiments of the disclosed subject matter.

FIG. 6 is a process flow diagram for an optimization process of a treatment plan using a single radiation field, according to various embodiments of the disclosed subject matter.

FIG. 7 is a process flow diagram for an optimization process of a treatment plan using multiple radiation fields, according to various embodiments of the disclosed subject matter.

### DETAILED DESCRIPTION

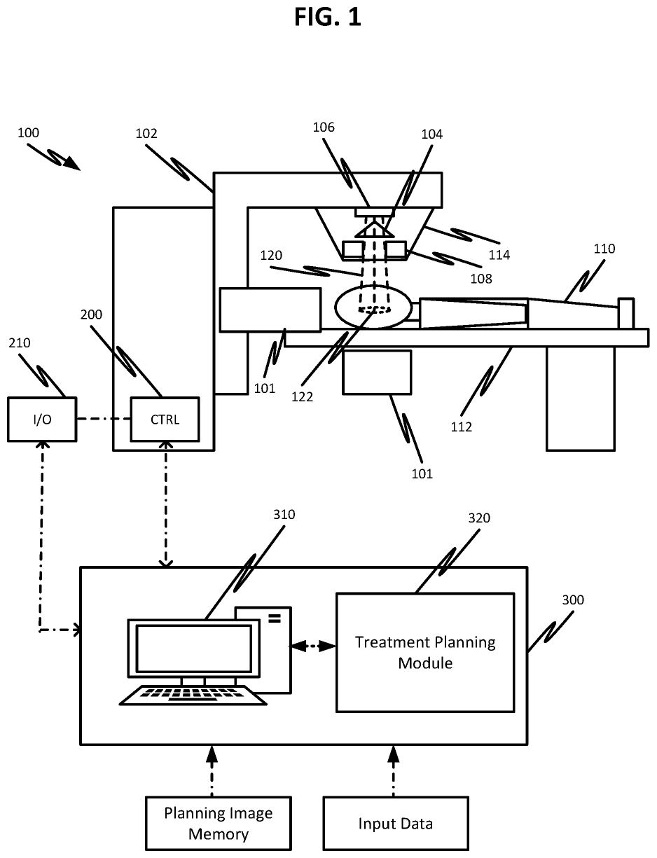

Referring to FIG. 1, an exemplary radiation therapy system 100 is shown, which can be used in radiation therapy, and which can deliver radiation in accordance with treatment plans that are determined using techniques described herein. The radiation therapy system 100 can provide radiation to a patient 110 positioned on a treatment couch 112 and can allow for the implementation of various radiation treatment protocols. The radiation therapy can include photon-based radiation therapy, particle therapy, electron beam therapy, or any other type of treatment therapy.

In an embodiment, the radiation therapy system 100 can include a radiation treatment device 101 such as, but not limited to, a LINAC operable to generate one or more beams of megavolt (MV) X-ray radiation for treatment. The LINAC may also be operable to generate one or more beams of kilovolt (kV) X-ray radiation, for example, for patient imaging. The system 100 has a gantry 102 supporting a radiation treatment head 114 with one or more radiation sources 106 and various beam modulation elements, such as, but not limited to, flattening filter 104 and collimating components 108. The collimating components 108 can include, for example, a multi-leaf collimator (MLC), upper and lower jaws, and/or other collimating elements. The collimating components 108 and/or the flattening filter 104 can be positioned within the radiation beam path by respective actuators (not shown), which can be controlled by controller 200.

The gantry 102 can be a ring gantry (i.e., it extends through a full 360° arc to create a complete ring or circle), but other types of mounting arrangements may also be employed. For example, a static beam, or a C-type, partial ring gantry, or robotic arm can be used. Any other framework capable of positioning the treatment head 114 at various rotational and/or axial positions relative to the patient 110 may also be used.

In an embodiment, the radiation therapy device is a MV energy intensity modulated radiation therapy (IMRT) device. The intensity profiles in such a system are tailored to the treatment requirements of the individual patient. The IMRT fields are delivered with MLC 108, which can be a computer-controlled mechanical beam shaping device attached to the head 114 and includes an assembly of metal fingers or leaves. For each beam direction, the optimized intensity profile is realized by sequential delivery of various subfields with optimized shapes and weights. From one subfield to the next, the leaves may move with the radiation beam on (i.e., dynamic multi-leaf collimation (DMLC)) or with the radiation beam off (i.e., segmented multi-leaf collimation (SMLC)). The MLC 108 therefore can be used to provide conformal treatment of tumors from various angles, as well as intensity modulated radiotherapy, whereby different radiation doses are delivered to different portions of the treatment area. The treatment volume, namely, the irradiated volume proximate to the isocenter in the path of the X-ray beam, is defined by the jaws, the head 114, and the MLC 108. In IMRT, the leaves of the MLC are moved so that the treatment volume comprises the volume exposed during the course of the treatment.

Alternatively, or additionally, the radiation therapy device 101 can be a tomotherapy device, a helical tomotherapy device, or a simplified intensity modulated arc therapy (SIMAT) device, a volumetric modulated arc therapy (VMAT) device, or a volumetric high-definition (or hyperarc) therapy (HDRT). In effect, any type of IMRT device can be employed as the radiation therapy device 101 of system 100, and can also include an on-board volumetric imaging, which can be used to generate in-treatment image data generated during a treatment session.

Alternatively, or additionally, the radiation therapy device 101 can be a proton therapy device, such as an intensity modulated proton therapy (IMPT) device (active scanning), where proton “pencil” beams originating from an accelerator are manipulated to treat tumors in layers of spots at varying depths by altering the number of protons, energy, and magnetic deflection. Unlike IMRT, where conformal dose distribution is obtained through the use of multiple beam arrangements (arcs) and multileaf collimators, IMPT relies on electromagnetic control of the pencil beam to achieve the target coverage while reducing radiation dose.

Alternatively, or additionally, the radiation therapy device 101 can be a proton therapy device, such as a passive scattered proton therapy (PSPT) device, where the proton beam is spread out to be a uniform beam through single or double scattering, and the edges of the beam can be laterally shaped by collimators to produce a uniform field distribution.

Each type of radiation therapy device can be accompanied by a corresponding radiation plan and radiation delivery procedure.

The controller 200, which can be, but is not limited to, a graphics processing unit (GPU), can include a computer with appropriate hardware such as a processor, and an operating system for running various software programs and/or communication applications. The controller 200 can include software programs that operate to communicate with the radiation therapy device 101, which software programs are operable to receive data from external software programs and hardware. The computer can also include any suitable input/output (I/O) devices 210, which can be adapted to allow communication between controller 200 and a user of the radiation therapy system 100, e.g., medical personnel. For example, the controller 200 can be provided with I/O interfaces, consoles, storage devices, memory, keyboard, mouse, monitor, printers, scanner, as well as a departmental information system (DIS) such as a communication and management interface (DICOM) for storing and transmitting medical imaging information and related data and enabling the integration of medical imaging devices such as scanners, servers, workstations, printers, network hardware, etc.

Alternatively, or additionally, the I/O devices 210 can provide access to a network (not shown) for transmitting data between controller 200 and remote systems. For example, the controller 200 can be networked via I/O 210 with other computers and radiation therapy systems. The radiation therapy system 100, the radiation treatment device 101, and the controller 200 can communicate with a network as well as databases and servers, for example, a dose calculation server (e.g., distributed dose calculation framework) and a treatment planning system 300. The controller 200 may also be configured to transfer medical image related data between different pieces of medical equipment.

The system 100 can also include a plurality of modules containing programmed instructions (e.g., as part of controller 200, or as separate modules within system 100, or integrated into other components of system 100), which instructions cause system 100 to perform different functions related to radiation therapy including adaptive radiation therapy or other radiation treatment, as discussed herein, when executed. For example, the system 100 can include or communicate with a treatment planning module 320 of a treatment planning system 300 operable to generate the treatment plan for the patient 110 based on a plurality of data input to the system by the medical personnel using computer 310, a patient positioning module operable to position and align the patient 110 with respect to a desired location, such as the isocenter of the gantry, for a particular radiation therapy treatment, an image acquiring module operable to instruct the radiation therapy system and/or the imaging device to acquire images of the patient 110 prior to the radiation therapy treatment (i.e., pre-treatment/reference images used for treatment planning and patient positioning) and/or during the radiation therapy treatment (i.e., in-treatment session images), and to instruct the radiation therapy system 100 and/or the imaging device 101 or other imaging devices or systems to acquire images of the patient 110.

The system 100 can further include one or more contour generation modules operable to generate contours of target volumes and other structures in pre-treatment (planning, reference) and in-treatment (treatment session) images, a contour verification module operable to verify a generated contours, a dose calculation module operable to calculate accumulated dose, a dose evaluation module operable to evaluate dose distribution within a desired volume, a bounding box generating module operable to automatically define a field bounding box for each individual radiation field, modules for electron density map generation, isodose distribution generation, dose volume histogram (DVH) generation, image synchronization, image display, treatment plan generation, treatment plan optimization, automatic optimization parameter generation, updating and selection, and adaptive directives and treatment information transfer. The system 100 can further include a treatment delivery module operable to instruct the radiation therapy device 100 to deliver a treatment plan to the patient 110.

The modules can be written in the C or C++ programming language, for example. Computer program code for carrying out operations as described herein may be written in any programming language, for example, C or C++ programming language.

The treatment planning system 300 can be used to generate optimized treatment plans for the radiation therapy system 100. The treatment planning system 300 includes program memories that contain processor executable instructions that, when executed by the processor 310, generate optimized treatment plans that can be executed by a processing unit (e.g., controller 200) of the radiation therapy system 100. The treatment planning system 300 is configured to communicate with a planning image memory containing image data, and with a knowledge base which is a database or other information retrieval system that contains plan templates including clinical goals (CGs) and priorities for different anatomical structures, as well as knowledge-based information such as patient records (i.e., previous/existing treatment plans) that are similar to the current patient record, treatment types, and knowledge-based statistical models, such as DVH models, for example.

Treatment planning generally starts with obtaining one or more planning images/image slices of the portion of the patient that includes the tumor. The images/image slices may be CT, CBCT, MRI, etc. images/image slices, for example. A qualified medical personnel (physician) next determines and delineates the treatment volume on the image/image slices. Delineating the treatment volume involves generating a body contour around the total volume to be irradiated, as well as delineating the structures contained within the body contour, such as, one or more malignant tumors that are to receive therapeutic doses of radiation (i.e., target volumes) and structures whose irradiation should be limited, since a dose of radiation in excess of a certain amount may adversely affect them (i.e., organs at risk (OARs)). The delineations are generally manually done on the planning image/image slices by the qualified medical personnel (physician), but the contouring may also be performed automatically or semiautomatically using any available segmentation algorithms. The planning images used for the treatment planning can also be images that were previously taken and stored in a planning image memory.

Typical delineations for the malignant tumor include the gross target volume (GTV), the clinical target volume (CTV), and the planning target volume (PTV). The (GTV) determines the anatomic region which harbors the highest tumor cell density and requires the highest prescribed dose. The (GTV) is the position and extent of the gross tumor, i.e., what can be seen, palpated or imaged. The (CTV) contains the (GTV), plus a margin for sub-clinical disease spread which therefore cannot be fully imaged. The (CTV) is the volume that must be adequately treated to achieve cure. The (PTV) allows for uncertainties in planning or treatment delivery. It is a geometric concept designed to ensure that the radiotherapy dose is actually delivered to the (CTV). The (PTV) is thus used to compensate for treatment setup uncertainties through volumetric expansion of the (CTV) margins. Although the treatment volume is a three-dimensional structure including the target volumes and other structures, for ease of illustration, the treatment volume is shown as a two-dimensional cross-sectional structure. This is not to be a limiting feature, however.

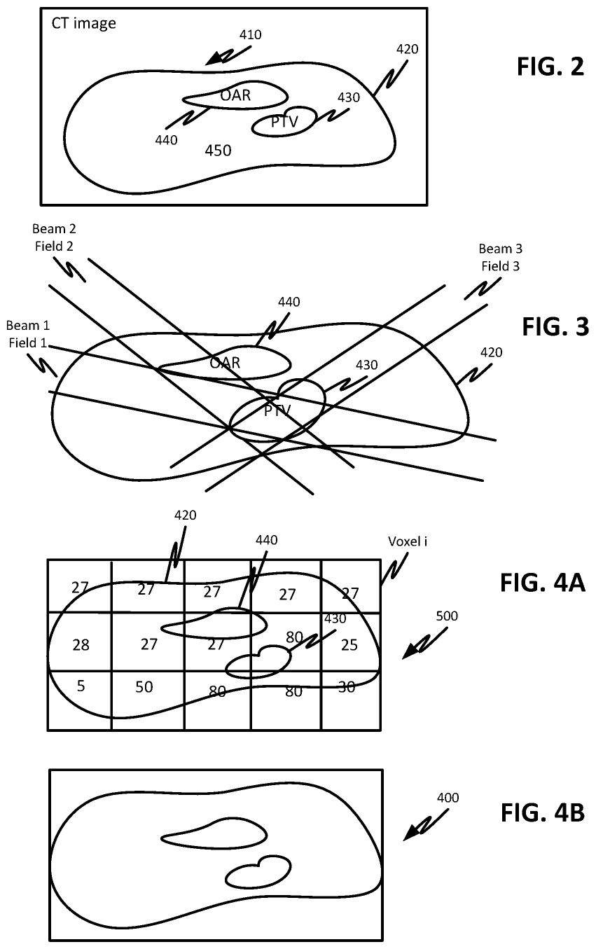

FIG. 2 illustrates an exemplary treatment volume 410 including the body contour 420, a target volume (PTV) 430, an OAR 440, as well as the other area 450 of the treatment volume 410 that is not the PTV 430 or the OAR 440 (i.e., the rest of the treatment volume 410).

After delineating the treatment volume 410, the physician specifies a preferred/desired dose distribution within the treatment volume 410. This can be expressed as a set or a template of clinical goals (CG). These clinical goals (CG) can be given, for example, in the form of mean dose of radiation (in Gray) to the target volume 430 and the dose that certain volume of an organ, such as an organ at risk (OAR) 440, or other areas 450 exposed to radiation within the treatment volume 410, must not exceed. Clinical goals, however, may also be given in other dimensions that are not in the form of dose of radiation to a target volume and dose to volume of organ, such as specifying dose values for each voxel within the treatment volume. Voxels represent the three-dimensional rectangular elements that the treatment volume is discretized into. During the treatment, each of these voxels will absorb a dose of radiation.

Each of the given goals can further be ordered in priority describing the importance of meeting a goal in comparison to another goal. Such a set is referred to as a prioritized set of clinical goals (prioritized CG). Each clinical goal can be expressed as a quality metric and its associated goal value. An exemplary prioritized set of clinical goals is:
GOAL 1: Target (PTV) must receive 90 Gy: Priority 1
GOAL 2: Organ at risk (OAR) must receive less than 25 Gy: Priority 2
GOAL 3: Other parts of the treatment volume must receive a mean dose of less than 30 Gy: Priority 3.

Another way of expressing desired dose distributions is by employing a library of clinically approved and delivered plans of previously treated patients with similar medical characteristics, in order to find a set of parameters for patient 110 that produces a clinically desirable plan. In this approach, an algorithm (i.e., a Dose Volume Histogram (DVH) model, for example) that has been trained from historical patient data (i.e., structures and dose distributions) is used as a starting point to predict the achievable dose distributions for a new set of patient structures. The achievable dose distributions are presented as a pair of Dose Volume Histograms (DVHs) representing the lower and upper bounds of the 95% confidence interval of the prediction. These DVH histograms can then be used as objectives for each structure within the treatment volume.

The physician also develops a set of treatment parameters that take into account constraints imposed on the treatment process by the radiation therapy system 100 used for delivering the radiation to the treatment volume 410. For example, the physician may need to consider the number of gantry angles needed to deliver the treatment beams at fixed directions (i.e., radiation fields), as well as any other parameters associated with each field, such as, but not limited to, MLC leaf positions, beam intensity profiles, etc.

In treatment planning for passive scattering (PSPT), the physician may need to consider the specific beam angles (i.e., gantry angle, treatment couch angle) along with an appropriate range and modulation of the spread out Bragg peak to ensure target volume coverage.

Based on the treatment parameters, a treatment plan is then generated. The generating of a treatment plan generally involves using an algorithm to solve the “inverse problem” of devising and optimizing a specific plan for irradiating the treatment volume 410 from a variety of angles or, in arc therapy, while the gantry is moving, to deliver the desired radiation dose to the target volume while minimizing irradiation of the nearby organs/tissues, etc., while taking into account the capabilities and limitations of the radiotherapy system 100. Thus, treatment planning essentially involves devising a plan to deliver an optimal dose distribution to the treatment volume. This may be reduced to a cost function (objective function) that accounts for various trade-offs inherent in such plans with constraints that must be met for the plan to be medically acceptable or physically possible.

Treatment planning for active scanning (IMPT) therapy also solves the inverse problem, except that for every beam it tries to position spots to achieve the desired dose to the target volume while sparing nearby organs at risk, with the option to uniformly cover the treatment volume with every beam (i.e., single field uniform dose) or to have multiple fields where the entirety of the target volume is covered by the multiple fields in the treatment plan, but not by a unique field in the treatment plan.

The algorithms that may be used for treatment planning are dose calculation algorithms based on a given set of parameters, such as gantry angle, MLC leaf positions, etc., or search algorithms which use various techniques to adjust the treatment parameters between dose calculations to achieve optimization of the plan. There are many algorithms that can be used for treatment planning. For example, dose calculation algorithms may include, but are not limited to, Monte Carlo techniques, pencil beam convolution, generalized Gaussian pencil beam, collapsed convolution, and anisotropic analytical algorithms. Search algorithms may include various stochastic and deterministic methods, including but not limited to, simulated annealing techniques, algebraic inverse treatment planning, simultaneous iterative inverse planning, iterative least-square inverse treatment planning, and superposition convolution, for example.

Each of these planning algorithms require iterative dose calculations for optimization and generally proceed by calculating the radiation dose received by each voxel in the treatment volume, adjusting one or more variable treatment parameters, such as the angle of irradiation or the positions of the MLC leaves, for example, and then recalculating the dose received by each voxel. This process is performed iteratively until an acceptable dose distribution is obtained, which then corresponds to the optimized plan.

To obtain an optimized treatment plan, the process starts with an initial or base dose calculation using starting parameters for developing the plan. For purposes of the present invention, the manner of arriving at the starting parameters is unimportant, so any of the variety of known methods for arriving at starting parameters is suitable. FIG. 3 illustrates an exemplary planning scheme including three beam fields (Beam 1-Beam 3), namely, the treatment plan calls for irradiating the treatment volume 410 from three different gantry angles using corresponding radiation beam fields defined by the MLC. Each incident beam (Beam 1-Beam 3) is divided into a collection of beamlets (x), each beamlet being a small rectangular element representing the discretized intensity profile of the beam within the field at a rectangular grid on the plane of the MLC aperture.

For this plan, the desired dose distribution specified by the physician is, for example: target (PTV) 430 must receive 90 Gy; organ at risk (OAR) 440 must receive less than 25 Gy; and other parts of the treatment volume 450 must receive a mean dose of less than 30 Gy.

In order to optimize the treatment plan, the dose distribution in the treatment volume 410 is calculated and compared to the desired dose distribution. Calculating the dose distribution within the treatment volume 410 involves calculating the dose absorbed at each voxel of the treatment volume 410 for each beamlet within a field. A standard linear model for calculating the dose absorbed at the ith voxel in the treatment volume can take the form:

$$d = Ax or \\ {d}_{i} = {\sum }_{j = 1}^{n} {a}_{ij} {x}_{j} , i = 1 , 2 , … , m$$

where x is a one-dimensional vector consisting of beamlet intensities of the contributing fields, aᵢⱼ represents the amount of dose absorbed by the ith voxel per unit intensity emission from the jth beamlet. The collection of values ay for all the voxels and beamlets forms a matrix A∈R^{m×n} known as the dose influence matrix or the kernel matrix. To compute the matrix A, and thus obtain the dose distribution for each voxel within the treatment volume 410 for all beamlets within each field, any of the previously mentioned dose distribution calculation algorithms can be used. For proton therapy, matrix A represents the dose distribution for each voxel within the treatment volume 410 for all individual spots within each field.

FIG. 4A illustrates an exemplary dose distribution grid 500 that has been superimposed on top of the treatment volume 410 after the dose distribution has been calculated using one of the above-mentioned dose calculation algorithms. The number within each box of the grid 500 represents the dose received by the corresponding voxel (Voxel i) of the treatment volume 410 for all beamlets (or spots) of all fields combined after an initial iteration of the dose calculation algorithm. The dose calculation volume 400 shown in FIG. 4B represents the volume containing all voxels (Voxel i) for which dose calculation has been done.

As illustrated in FIG. 4A, the dose in the target volume (PTV) 430 is 80 Gy, which is less than the desired dose, the dose in the OAR 440 is 27 Gy, which is higher than the desired dose, and the dose in some of the other areas 450 of the body contour 420 is more than the desired dose. If the dose distribution does not conform to the desired dose distribution within a predetermined threshold (preset by the physician), the plan is not optimal. In such a case, one or more treatment parameters can be modified, and the dose distribution recalculated using any of the previously mentioned dose calculation algorithms. This process is ideally performed iteratively until the desired dose distribution is obtained, at which time the plan is said to be optimized. However, the amount of time needed to perform the large number of calculations for each iteration places a practical limit on the number of iterations that can be performed. As such, generally, the optimization process is terminated after a predetermined number of iterations, or after some other practical limit is reached. Due to this, there is a tradeoff between the accuracy and the speed of the different algorithms used for treatment planning.

Since the treatment planning algorithms do calculate the radiation dose received by each voxel (Voxel i) in the treatment volume 410, the computation effort, and thus the execution time of the treatment optimization process, is directly dependent on the number of voxels for which dose calculation is performed. In other words, the computation time is directly dependent on the kernel size A and thus the size of the dose calculation volume 400.

In accordance with the present invention, a method is disclosed wherein the dose calculation volume 400 is reduced. Reducing the dose calculation volume reduces the number of voxels for which dose calculation is performed at each iteration of the optimization process. This results in a faster optimization process, and thus a faster convergence toward the plan objectives.

In embodiments, the dose calculation volume is reduced by cropping the dose calculation volume 400 associated with the treatment volume 410 using a field bounding box. In specific embodiments, the dose calculation volume 400 is reduced to its minimum size.

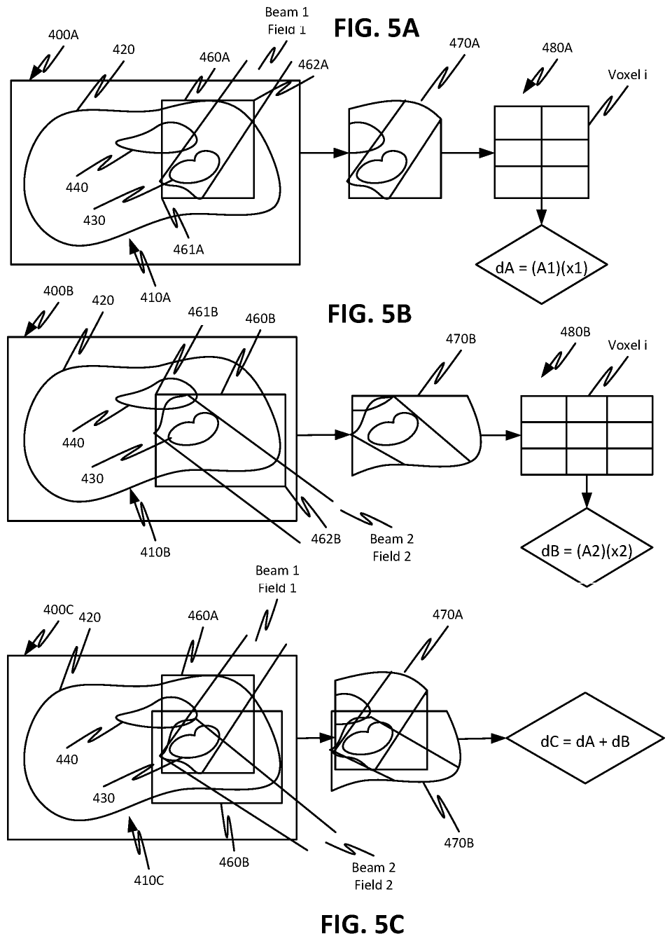

FIGS. 5A-5C illustrate exemplary processes for cropping a dose calculation volume (400A-400C). The cropping can be done by employing constraints/limitations on the dose calculation algorithms. These constraints can be derived from bounding box annotations.

For example, as shown in FIG. 5A, for a treatment plan that calls for irradiating the treatment volume 410A using a single beam Field 1, without cropping, the treatment plan optimization would require iterative calculation of dose distribution for every voxel within the dose calculation volume 400A corresponding to the treatment volume 410A. To reduce the size of the dose calculation volume 400A, a limitation can be applied to the dose calculation algorithm to only use the voxels contained within a limited volume. This volume can be defined using the spatial constraints of a bounding box 460A. A bounding box is an imaginary rectangle that acts to define an object and can generally be defined using its spatial coordinates, such as the top and bottom opposing corner coordinates (i.e., to top right corner and bottom left corner of the rectangle, for example). In this case, a bounding box is used to define a smaller volume within the dose calculation volume 410A. Bounding box 460A can be defined by specifying corner coordinates 461A and 462A, for example, with corner 461A being the corner defined by the intersection of the radiation Field 1 with the target volume 430, and corner 462A being defined by the intersection of the Field 1 with the body contour 420. The box 460A created using these corners 461A, 462A, defines a field bounding box, as it focuses on delineating a volume around the interaction of the Field 1 with the target volume 430. The spatial coordinates of the field bounding box 460A may be set by a user based on the coordinates of the pixels of the image containing the treatment volume and the beam geometry.

The field bounding box 460A is then used to crop the dose calculation volume 400A at the intersection of the field bounding box 460A with the body contour 420. By cropping the dose calculation volume 400A at the intersection of the field bounding box 460A with the body contour 420, a cropped volume 470A is obtained. The dose calculation volume 480A corresponding to the cropped volume 470A is the minimum dose calculation volume. The number of voxels (Voxel i) included in the dose calculation volume 480A is less than the number of voxels included in the dose calculation volume 400A.

The spatial constraints associated with the field bounding box 460A can be applied to the dose calculation algorithm as a spatial constraint/limitation. By including such a limitation, the dose calculation algorithm will be limited to considering only the voxels (Voxel i) that are included in the minimum dose calculation volume 480A. Thus, to optimize the treatment plan, at each iteration, the computation is for matrix A₁, which is limited to calculating dose absorption by each voxel within the calculation volume 480A for all beamlets within Field 1. The dose distribution da therefore can be calculated using the influence matrix A₁ for field beamlets x₁:d_{A}=(A₁)(x₁). Since the kernel size A₁ for the dose distribution calculation volume is reduced from the original kernel size A, the computation time for solving for A₁ is reduced.

FIG. 5B illustrates applying a field bounding box 460B defined by a different beam Field 2. As in FIG. 5A, the treatment plan calls for irradiating the treatment volume 410B using a single beam Field 2. Without cropping the treatment volume 410B, the treatment plan optimization would require iterative calculation of dose distribution for every voxel within the dose calculation volume 400B. To reduce the size of the dose calculation volume 400B, a limitation can be applied to the dose calculation algorithm to only use the voxels contained within a limited volume for dose calculation. This volume can be defined using a field bounding box 460B. The field bounding box 460B can be defined with two corner coordinates 461B and 462B, with corner 461B being the corner where the radiation Field 2 intersects with the target volume 430, and corner 462B being the corner where the Field 2 intersects with the body contour 420. The box 460B created using these two opposing corners 461B, 462B, defines a field bounding box, as it focuses on defining a box around the interaction of the Field 2 with the target volume 430 within the body contour 420.

The field bounding box 460B is then used to crop the dose calculation volume 400B at the intersection of the field bounding box 460B with the body contour 420. By cropping the dose calculation volume 400B at the intersection of the field bounding box 460B with the body contour 420, a cropped treatment volume 470B is obtained. The dose calculation volume 480B corresponding to the cropped treatment volume 470B is the minimum dose calculation volume. The number of voxels (Voxel i) included in the dose calculation volume 480B is less than the number of voxels included in the dose calculation volume 400B.

The spatial constraints associated with the field bounding box 460B can be applied to the dose calculation algorithm as a spatial constraint/limitation. By including such a limitation, the dose calculation algorithm will be limited to considering only the voxels (Voxel i) that are included in the minimum dose calculation volume 480B. Thus, to optimize the treatment plan, at each iteration, the computation is for matrix A₂, which is limited to calculating dose absorption by each voxel within the calculation volume 480B for all beamlets within Field 2. The dose distribution de therefore can be calculated using the influence matrix A₂ for field beamlets x₂:d_{B}=(A₂)(x₂). Since the kernel size A₂ for the dose distribution calculation volume is reduced from the original kernel size A, the computation time for solving for A₂ is reduced.

FIG. 5C illustrates a case where the treatment plan calls for irradiating the treatment volume 410 from both fields Field 1 and Field 2. In such a case, the field bounding box 460A associated with Field 1 is used to crop the dose calculation volume 400C at the intersection of the field bounding box 460A with the body contour 420 to obtain the dose calculation volume 480A, and the field bounding box 460B associated with Field 2 is used to crop the dose calculation volume 400C at the intersection of the field bounding box 460B with the body contour 420 to obtain the dose calculation volume 480B.

For each cropped dose calculation volume (480A, 480B), the corresponding dose distributions (A₁, A₂) are calculated. For example, matrix A₁ is calculated for Field 1 and matrix A₂ is calculated for Field 2. The total dose distribution dc for the full treatment plan is reconstructed by summing the individual cropped field doses da and de for the cropped dose calculation volumes: d_{C}=d_{A}+d_{B}=(A₁)(x₁)+(A₂)(x₂).

The number of fields used in the example of FIG. 5C is exemplary only. The cropping and optimization methods described herein can be applied for any number of fields included in the treatment plan. Thus, for a treatment plan containing a plurality of fields, the dose distribution of the full treatment plan is reconstructed by summing the individual field doses cropped to their respective bounding boxes: d=Σᵢ₌₁ⁿ dᵢ

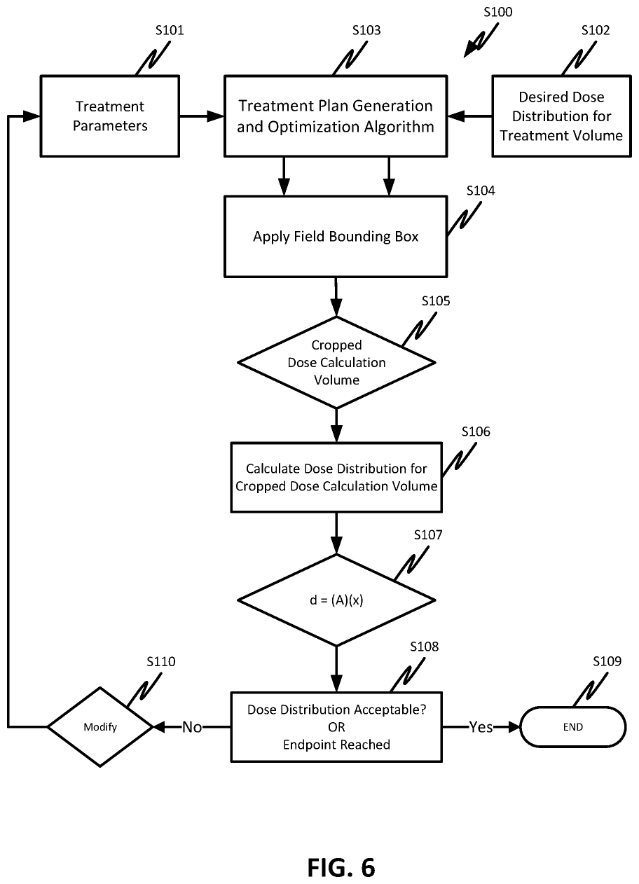

FIG. 6 is a flow diagram showing the steps of an embodiment of a treatment plan optimization process S100 of the instant invention. In steps S101 and S102, the treatment parameters as well as the desired dose distribution for a treatment volume are used as inputs to a treatment planning and optimization module. As previously discussed, the treatment parameters include parameters that take into consideration constraints imposed on the treatment process by the radiation therapy system used for delivering the radiation to a treatment volume. These parameters may include, but are not limited to, parameters related to gantry angles, MLC configurations, beam intensity profiles, etc. For example, the treatment parameters may specify the number of fields (5 fields, for example), gantry angles (at 0°, 60°, 90°, 230°, and 280°, for example), number of beamlets within each field (10×15 beamlets, for example), and the size of each beamlet (1×1 cm, for example) for delivering the desired dose to the treatment volume. Process S100 illustrates a treatment plan optimization process where a single field is used.

The treatment volume may be a body contour including delineated target volumes and organs at risk on a CT image, for example. The desired dose distribution can be defined using clinical goals, prioritized clinical goals, or any other way as described throughout this specification.

The treatment plan generation and optimization algorithm S103 is configured to calculate a dose distribution within a reduced dose calculation volume at each iteration of the treatment plan optimization process. The volume is reduced by applying a spatial constraint/limitation on the dose calculation algorithm derived from the spatial coordinates of a field bounding box S104. The spatial coordinates of the field bounding box can be defined as the corners that bound the field interaction with the target volume within the treatment volume. By applying a field bounding box at S104 that crops the treatment volume at the intersection of the field bounding box with the body contour of the treatment volume, all voxels that are outside the field bounding box are disregarded. The plan generation and optimization algorithm S103 therefore will only calculate and evaluate the dose (beamlet dose for radiotherapy and individual spot dose for proton therapy) of the voxels included in the cropped dose calculation volume of S105, and will disregard the voxels contained in the rest of the treatment volume.

In S106, the dose distribution for the voxels in the cropped dose calculation volume is calculated. The dose distribution S107 obtained for all voxels in the cropped dose calculation volume S105 for all beamlets contained in the single radiation field is then compared to the desired dose distribution in S108. If the obtained dose distribution is not within a predetermined threshold of the desired dose distribution, one or more of the treatment parameters can be modified/adjusted in S110, and the process S100 is repeated until either the dose distribution in S108 is acceptable or until a different endpoint is reached in S108. The different endpoint in S108 may be the number of iterations of process S100, or the elapse of a predetermined computation time. At that point, the process S100 ends at S109, and the so obtained treatment plan can be considered to be the optimal treatment plan for irradiating the treatment volume, so that the target volume (i.e., the tumor) receives a prescribed dose while limiting the irradiation of the adjacent healthy tissue and organ at risk to acceptable limits. This optimal treatment plan can then be applied to irradiate the patient 110 using the radiation treatment system 100.

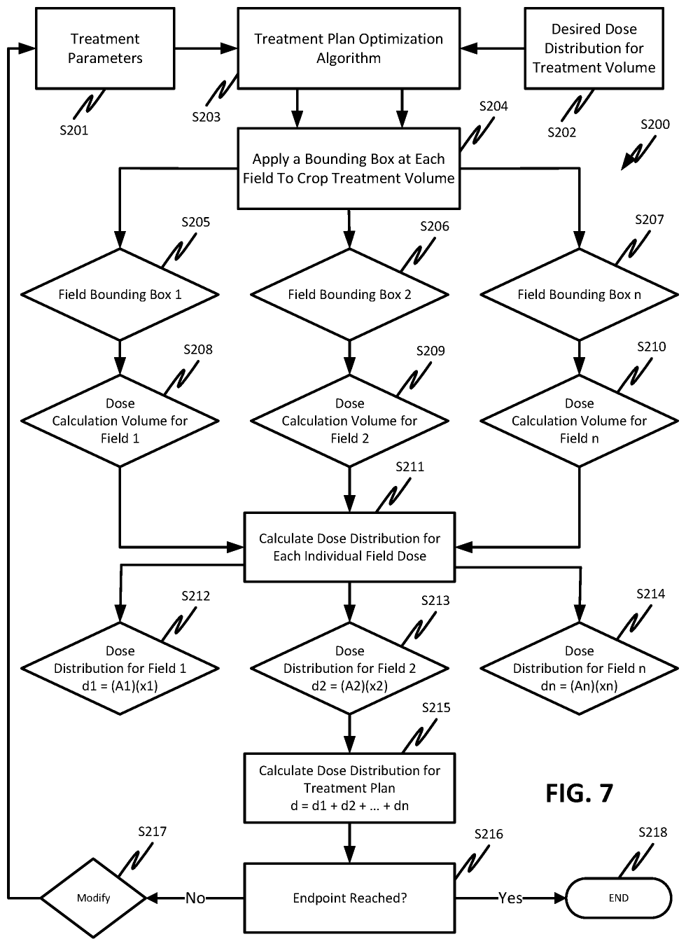

FIG. 7 is a flow chart showing the steps of another embodiment of a treatment plan optimization process S200 of the instant invention. In steps S201 and S202, the treatment parameters as well as the desired dose distribution for a treatment volume are used as inputs to a treatment planning and optimization module. As previously discussed, the treatment parameters include parameters that take into consideration constraints imposed on the treatment process by the radiation therapy system used for delivering the radiation to a treatment volume. These parameters may include, but are not limited to, parameters related to gantry angles, MLC configurations, beam intensity profiles, etc. The treatment parameters may specify the number of fields (5 fields, for example), gantry angles (at 0°, 60°, 90° 230°, and 280°, for example), number of beamlets within each field (10×15 beamlets, for example), and the size of each beamlet (1×1 cm, for example) for delivering the desired dose to the treatment volume. Process S200 illustrates a treatment plan optimization process where a plurality of fields (Field 1-Field n) are used, each field containing its own number of beamlets.

The treatment volume may be a body contour including delineated target volumes and organs at risk on a CT image, for example. The desired dose distribution can be defined using clinical goals, prioritized clinical goals, or any other way as described throughout this specification.

By applying a corresponding field bounding box in S204, the treatment plan generation and optimization algorithm S203 is configured to calculate a dose distribution within each reduced dose calculation volume obtained for each individual field. The dose calculation volume is reduced by applying a spatial constraint/limitation on the dose calculation algorithm derived from the spatial coordinates of each field bounding box applied in S204. The spatial coordinates of a first field bounding box S205 can be defined as the corners that bound the first field's (Field 1) interaction with the target volume within the treatment volume. The spatial coordinates of a second field bounding box S206 can be defined as the corners that bound the second field's (Field 2) interaction with the target volume within the treatment volume. The spatial coordinates of the nth field bounding box S207 can be defined as the corners that bound the nth field's (Field n) interaction with the target volume within the treatment volume.

By applying a corresponding field bounding box (S205-S207) for each field of the treatment plan, the treatment volume is cropped at the intersection of each individual field bounding box (S205-S207) with the body contour of the treatment volume. This results in corresponding cropped dose calculation volumes S208-S210. The plan generation and optimization algorithm S203 will then calculate for each field individually the dose distribution within the corresponding cropped dose calculation volumes S208-S210 in S211. The dose distribution obtained for Field 1 (S212), the dose distribution obtained for Field 2 (S213), and the dose distribution obtained for Field n (S214) can be added in S215 to obtain the dose distribution for all fields (Field 1-Field n) of the treatment plan. The dose distribution obtained in S215 is then compared to the desired dose distribution in S216. If the obtained dose distribution is not within a predetermined threshold of the desired dose distribution, one or more of the treatment parameters can be modified/adjusted in S217, and the process S200 is repeated until either the dose distribution in S216 is acceptable or until a predetermined endpoint is reached in S216. The endpoint in S216 may be set as a preset number of iterations of process S200, or the elapse of a predetermined computation time. When that is reached in S216, the process S200 ends at S218, and the so obtained treatment plan is the optimal treatment plan for irradiating the treatment volume so that the target volume (i.e., the tumor) receives a prescribed dose while the irradiation of the adjacent healthy tissues and organs at risk are at acceptable limits. This optimal treatment plan can then be used to irradiate the patient 110 using the radiation treatment system 100.

Software stored in the planning image memory and/or computer 310 of the treatment planning system 300 is configured to be loaded and processed in any conventional manner, and is configured to be executed in order to optimize a treatment plan according to the process steps described throughout this specification. The software is further configured to optimize the treatment plan for irradiating a target volume using a radiotherapy system having a multileaf collimator and is capable of irradiating the treatment volume from one or a plurality of angles. The treatment planning software includes at least one dose calculation algorithm for calculating dose distribution for a dose calculation volume that is cropped using a corresponding field bounding box. The treatment planning software further includes software that is configured to automatically recognize the spatial constraints applied by one or more field bounding boxes and crop the dose calculation volume according to the constraints imposed by each of the field bounding boxes. The treatment planning software further includes software for reconstructing the dose distribution of the full treatment plan by summing the individual cropped field doses.

The treatment planning software further includes software for translating the results of the optimized treatment plan into instructions for operating the radiation therapy system 100 for controlling the leaves of the MLC and the angle of irradiation in order to deliver the optimized radiation treatment plan to the patient 110.

It is thus apparent that methods and systems are disclosed herein where in order to reduce the computation time for dose calculation at each iteration of a treatment plan optimization process, the size of the dose calculation volume is reduced at each field to its minimum, and the dose distribution is calculated individually for each field. The dose distribution for the treatment plan can then be calculated by adding the individual dose fields obtained.

It is also apparent that systems including a computer processing device configured to execute a sequence of programmed instructions embodied on a computer-readable storage medium, the execution thereof causing the system to execute any or alternatively a combination of any of the method steps disclosed herein, are also disclosed.

A non-transitory computer-readable storage medium upon which is embodied a sequence of programmed instructions for the generation of reduced dose distribution volumes using field bounding boxes, and a computer processing system that executes the sequence of programmed instructions embodied on the computer-readable storage medium are also disclosed. Execution of the sequence of programmed instructions can cause the computer processing system to execute the treatment plan optimization processes described herein.

It will be appreciated that the aspects of the disclosed subject matter can be implemented, fully or partially, in hardware, hardware programmed by software, software instruction stored on a computer readable medium (e.g., a non-transitory computer readable medium), or any combination of the above.

For example, components of the disclosed subject matter, including components such as a controller, process, or any other feature, can include, but are not limited to, a personal computer or workstation or other such computing system that includes a processor, microprocessor, microcontroller device, or is comprised of control logic including integrated circuits such as, for example, an application specific integrated circuit (ASIC).

Features discussed herein can be performed on a single or distributed processor (single and/or multi-core), by components distributed across multiple computers or systems, or by components co-located in a single processor or system. For example, aspects of the disclosed subject matter can be implemented via a programmed general purpose computer, an integrated circuit device, (e.g., ASIC), a digital signal processor (DSP), an electronic device programmed with microcode (e.g., a microprocessor or microcontroller), a hard-wired electronic or logic circuit, a programmable logic circuit (e.g., programmable logic device (PLD), programmable logic array (PLA), field-programmable gate array (FPGA), programmable array logic (PAL)), software stored on a computer-readable medium or signal, an optical computing device, a networked system of electronic and/or optical devices, a special purpose computing device, a semiconductor chip, a software module or object stored on a computer-readable medium or signal.

When implemented in software, functions may be stored on or transmitted over as one or more instructions or code on a computer-readable medium. The steps of a method or algorithm disclosed herein may be embodied in a processor-executable software module, which may reside on a computer-readable medium. Instructions can be compiled from source code instructions provided in accordance with a programming language. The sequence of programmed instructions and data associated therewith can be stored in a computer-readable medium (e.g., a non-transitory computer readable medium), such as a computer memory or storage device, which can be any suitable memory apparatus, such as, but not limited to read-only memory (ROM), programmable read-only memory (PROM), electrically erasable programmable read-only memory (EEPROM), random-access memory (RAM), flash memory, disk drive, etc.

As used herein, computer-readable media includes both computer storage media and communication media, including any medium that facilitates transfer of a computer program from one place to another. Thus, a storage media may be any available media that may be accessed by a computer. By way of example, and not limitation, such computer-readable media may comprise RAM, ROM, EEPROM, CD-ROM or other optical disk storage, magnetic disk storage or other magnetic storage devices, or any other medium that may be used to carry or store desired program code in the form of instructions or data structures and that may be accessed by a computer.

Also, any connection is properly termed a computer-readable medium. For example, if the software is transmitted from a website, server, or other remote source using a transmission medium (e.g., coaxial cable, fiber optic cable, twisted pair, digital subscriber line (DSL), or wireless technologies such as infrared, radio, and microwave), then the transmission medium is included in the definition of computer-readable medium. Moreover, the operations of a method or algorithm may reside as one of (or any combination of) or a set of codes and/or instructions on a machine readable medium and/or computer-readable medium, which may be incorporated into a computer program product.

One of ordinary skill in the art will readily appreciate that the above description is not exhaustive, and that aspects of the disclosed subject matter may be implemented other than as specifically disclosed above. Indeed, embodiments of the disclosed subject matter can be implemented in hardware and/or software using any known or later developed systems, structures, devices, and/or software by those of ordinary skill in the applicable art from the functional description provided herein.

In this application, unless specifically stated otherwise, the use of the singular includes the plural, and the separate use of “or” and “and” includes the other, i.e., “and/or.” Furthermore, use of the terms “including” or “having,” as well as other forms such as “includes,” “included,” “has,” or “had,” are intended to have the same effect as “comprising” and thus should not be understood as limiting.

Any range described herein will be understood to include the endpoints and all values between the endpoints. Whenever “substantially,” “approximately,” “essentially,” “near,” or similar language is used in combination with a specific value, variations up to and including 10% of that value are intended, unless explicitly stated otherwise.

The terms “system,” “device,” and “module” have been used interchangeably herein, and the use of one term in the description of an embodiment does not preclude the application of the other terms to that embodiment or any other embodiment.

Many alternatives, modifications, and variations are enabled by the present disclosure. While specific examples have been shown and described in detail to illustrate the application of the principles of the present invention, it will be understood that the invention may be embodied otherwise without departing from such principles. For example, disclosed features may be combined, rearranged, omitted, etc. to produce additional embodiments, while certain disclosed features may sometimes be used to advantage without a corresponding use of other features. Accordingly, Applicant intends to embrace all such alternative, modifications, equivalents, and variations that are within the spirit and scope of the present invention.

1. A method for optimizing a treatment plan for delivering a radiation dose to a treatment volume within a patient from one or more radiation fields, comprising:
for each radiation field, obtaining a reduced volume for which dose distribution is calculated;
calculating a dose distribution for each reduced dose calculation volume; and
calculating a dose distribution for the treatment plan by summing the calculated dose distributions for each individual radiation field.

2. The method of claim 1, wherein obtaining a reduced volume for a radiation field includes applying a corresponding field bounding box to crop the treatment volume.

3. The method of claim 2, wherein the treatment volume is a body structure containing at least a target volume and an organ at risk.

4. The method of claim 3, wherein the cropping includes defining the corresponding field bounding box to enclose the target volume and cropping the treatment volume at the intersection of the field bounding box with the body structure.

5. The method of claim 4, wherein the calculating of the dose distribution for a cropped treatment volume includes calculating absorbed dose of radiation at each voxel included in the cropped treatment volume.

6. A method for optimizing a treatment plan for delivering a prescribed radiation dose to a predefined treatment volume within a patient using a radiation beam delivery system, the method comprising:
receiving information related to the prescribed radiation dose, the predefined treatment volume, and a plurality of parameters associated with the radiation beam delivery system, the plurality of parameters including information regarding radiation beam fields; and
applying a treatment plan optimization algorithm to calculate dose distribution within the treatment volume based on the received information by:
reducing a dose calculation volume at each radiation field;
calculating a dose distribution for each reduced dose calculation volume; and
adding the dose distributions obtained for each of the reduced dose calculation volumes to obtain the dose distribution for the treatment plan.

7. The method of claim 6, wherein the treatment volume is a body structure containing at least a target volume.

8. The method of claim 7, wherein the reducing of the dose calculation volume at each radiation field includes defining a field bounding box for each radiation field and cropping the treatment volume at the intersection of the bounding box with the body structure.

9. The method of claim 8, wherein the calculating of the dose distribution for a cropped treatment volume includes calculating absorbed dose of radiation at each voxel included in the cropped treatment volume.

10. A system for developing a treatment plan for the delivery of a prescribed radiation dose to a treatment volume within a patient, comprising:
a processor; and
a memory coupled to the processor, the memory including instructions that when executed by the processor cause the processor to:
receive information related to the prescribed radiation dose, the treatment volume, and a plurality of parameters associated with the radiation beam delivery system, the plurality of parameters including information regarding radiation beam fields;
develop a treatment plan optimization model based on the received information, the treatment plan optimization model being configured to find an optimal dose distribution within the treatment volume for each radiation beam field; and
generate an optimal treatment plan based on the treatment plan optimization model,
wherein the developing of the treatment plan optimization model includes restricting the treatment volume for which the dose distribution is determined at each iteration of the optimization algorithm.

11. The system of claim 10, wherein the restricting of the treatment volume includes cropping, for each radiation field, the treatment volume for which radiation dose is calculated.

12. The system of claim 11, wherein the cropping includes defining a bounding box for the treatment volume for each radiation field and cropping the treatment volume at the intersection of the respective bounding box with the treatment volume.

13. The system of claim 12, wherein the calculating of the dose distribution for a cropped treatment volume includes calculating absorbed dose of radiation at each voxel included in the cropped treatment volume.

14. The system of claim 13, further comprising calculating dose distribution for the treatment volume by summing the dose distributions obtained for each of the cropped treatment volumes.

15. A treatment planning system, comprising:
a user interface; and
a treatment planning module configured to generate a treatment plan for delivering a prescribed radiation dose to a treatment volume within a patient from one or more radiation fields based on prescribed clinical goals received via the user interface,
wherein the treatment planning module optimizes the treatment plan by:
calculating dose distribution within the treatment volume at each iteration of the optimization process;
determining whether the calculated dose distribution is within an acceptable threshold of the desired dose distribution;
modifying one or more treatment parameters and repeating the optimization process until a predetermined endpoint is reached,
wherein the calculating of the dose distribution includes:
determining dose distribution individually for each field; and
summing the dose distributions obtained for each individual field, and wherein the determining dose distribution individually for each field includes:
cropping the treatment volume to a minimum volume; and
calculating the dose distribution for the corresponding minimum volume.

16. The treatment planning system of claim 15, wherein the cropping of the treatment volume includes applying a corresponding field bounding box.

17. The treatment planning system of claim 16, wherein the cropping includes cropping the treatment volume at the intersection of the field bounding box with the treatment volume.

18. The system of claim 18, wherein the calculating of the dose distribution for a cropped treatment volume includes calculating absorbed dose of radiation at each voxel included in the cropped treatment volume.

19. A non-transitory computer-readable storage medium having treatment planning software stored thereon, the treatment planning software including software for optimizing a treatment plan for irradiating a treatment volume using a radiation therapy system, the treatment planning software comprising:
instructions for causing the radiation system to define a field bounding box for each dose field of the treatment plan;
instructions for causing the radiation system to reduce a dose calculation volume associated with each individual dose field using the correspondingly defined field bounding box;
instructions for causing the radiation system to calculate dose distributions for each of the reduced calculation volumes; and
instructions for causing the radiation system to sum the individual dose distributions to obtain the dose distribution for the treatment plan.

20. The non-transitory computer-readable storage medium of claim 19, wherein the optimizing is iteratively performed until a predetermined endpoint is reached.
